(19) United States
(12) Patent Application Publication
(10) Pub. No.: US 2023/0089786 A1
(43) Pub. Date: Mar. 23, 2023

(54) LASER ABLATION CATHETER WITH OUTER JACKET SUPPORT

(71) Applicant: RA MEDICAL SYSTEMS, INC., Carlsbad, CA (US)

(72) Inventors: Brian MARSH, Carlsbad, CA (US); James B. LAUDENSLAGER, San Marcos, CA (US)

(21) Appl. No.: 17/801,764

(22) PCT Filed: Feb. 23, 2021
(86) PCT No.: PCT/US2021/019199

§ 371 Date: Aug. 23, 2022

Related U.S. Application Data
(60) Provisional application No. 62/980,968, filed on Feb. 24, 2020.

(51) Int. Cl. A61B 18/24

(52) U.S. Cl. CPC A61B 18/245; A61B 2018/00166

(57) ABSTRACT

Outer jacket support embodiments for laser ablation catheter embodiments are discussed herein. In some cases, such outer jacket support embodiments may be useful for improving the robustness and performance of laser ablation catheters.

### RELATED APPLICATIONS

This application is a national stage application under 35 U.S.C. section 371, which claims the benefit of priority to PCT Application No. PCT/US2021/019199, filed Feb. 23, 2021, by Brian MARSH et al., and titled “LASER ABLATION CATHETER WITH OUTER JACKET SUPPORT,” which claims priority from U.S. provisional patent application serial number 62/980,968 filed Feb. 24, 2020, by Brian MARSH et al., and titled “LIQUID FILLED LASER ABLATION CATHETER WITH FULL LENGTH OUTER JACKET SUPPORT”, each of which is incorporated by reference herein in its entirety.

### BACKGROUND

During catheter procedures, which may include angioplasty or atherectomy, medical catheters often pass through tortuous paths of body lumens. During passage various forces may need to be applied to the medical catheter to deliver it to a target site and perform a desired procedure. In some cases, a medical catheter may be inserted into a patient's body through a hemostatic support catheter, and this can result in relative friction between the support catheter and the medical catheter and therefore increase the force required to advance the medical catheter to a target site. In addition, medical catheters sometimes include fragile components such as fiber optics, electrical components, thin walled tubing or the like. As such, when a user of a medical catheter is performing a procedure, the user can sometimes deform a shaft of the medical catheter while applying the forces required to advance the medical catheter to the target site. Such deformation may, in turn, lead to reduced performance of the medical catheter in certain circumstances. What have been needed are devices and methods which allow a user of a medical catheter to position the medical catheter at a target site without deforming the medical catheter shaft or components disposed within the shaft of the medical catheter. What have been needed are devices and methods that allow a medical catheter having a liquid filled waveguide to withstand the forces required to position the medical catheter while minimizing undesired bulk to the outer profile of the medical catheter.

### SUMMARY

Some embodiments of a laser ablation catheter that may be used to ablate blockages as well as performing atherectomy in body lumens using high energy and high-power ultraviolet (UV) laser pulses may include a liquid filled waveguide. The liquid filled waveguide may include a catheter tube having an inner layer with a first index of refraction and an optical fluid that may be biocompatible and ultraviolet light transparent disposed within and completely filling an inner lumen of the catheter tube. The optical fluid may have a second index of refraction which is greater than the first index of refraction. The laser ablation catheter may also include a distal optical window which may be ultraviolet grade and elongated in configuration and which may be disposed in liquid sealed relation to a surface of the catheter tube at a distal end of the catheter tube. The distal optical window may also be disposed in optical communication with the optical fluid. The laser ablation catheter may also include a proximal optical window which is disposed in a liquid sealed relation to a surface of the catheter tube at a proximal end of the catheter tube. The proximal optical window may also be in optical communication with the optical fluid. The laser ablation catheter embodiment may further include an outer jacket that is disposed over the outer surface of the catheter tube with a proximal end that is disposed at a proximal portion of the catheter tube and a distal end that is disposed at a distal end of the catheter tube. The outer jacket may be secured relative to the catheter tube such that the outer jacket remains substantially fixed along a longitudinal axis thereof relative to the catheter tube. The outer jacket may also have a tubular configuration having an inner lumen with an inner surface, with the inner surface of the inner lumen being configured to slide over an exterior surface of the catheter tube with a close fit therebetween. The outer jacket may also include a tubular jacket body that includes an inner jacket layer comprised of a first material and an outer jacket layer comprised of a second material with the second material being different than the first material. The outer jacket may optionally have a longitudinal stiffness that is greater than a longitudinal stiffness of the catheter tube at the proximal portion of the catheter tube. The outer jacket may also include a reinforcement which is crush resistant and which may optionally be disposed between the inner jacket layer and the outer jacket layer of the tubular jacket body. In some cases, the tubular jacket body and reinforcement may be configured to increase the stiffness and crush resistance of the laser ablation catheter.

Some embodiments of a laser ablation catheter may include a liquid filled waveguide having a catheter tube with an inner layer with a first index of refraction and an optical fluid disposed within and completely filling an inner lumen of the catheter tube. The optical fluid may have a second index of refraction which is greater than the first index of refraction. A distal optical window may be disposed in optical communication with the optical fluid and disposed in sealed relation to an inner surface of the catheter tube at a distal end of the catheter tube. The liquid filled waveguide may also include a proximal optical window which is disposed in optical communication with the optical fluid and which is also disposed in a liquid sealed relation to the inner surface of the catheter tube at a proximal end of the catheter tube. The laser ablation catheter embodiment may also include an outer jacket which is disposed over an outer surface of the catheter tube. The outer jacket may have a proximal end that is disposed at a proximal portion of the catheter tube and a distal end that is disposed at a distal end of the catheter tube. In some instances, the outer jacket may include a tubular configuration having an inner lumen with an inner surface. The inner surface of the inner lumen of the outer jacket may be configured to slide over an outer surface of the catheter tube with a close fit therebetween. The outer jacket may also have a tubular jacket body that includes a longitudinal stiffness greater than a proximal portion of the catheter tube. The tubular jacket body may also include an inner jacket layer comprised of a first material and an outer jacket layer comprised of a second material. The outer jacket may also have a reinforcement that is crush resistant and disposed between the first layer and second layer of the tubular jacket body. An eccentric guidewire lumen may be disposed adjacent an outer surface of the outer jacket. For some embodiments, the eccentric guidewire lumen may have a proximal port and a distal port. The distal port may be disposed adjacent the distal end of the laser ablation catheter. The laser ablation catheter may also have a distal housing with a primary lumen which has a longitudinal axis that is concentric with a longitudinal axis of the catheter tube. The primary lumen may also be configured to accommodate the distal optical window. The distal housing may also have an eccentric passage which is disposed adjacent the primary lumen and which is configured to accommodate the eccentric guidewire lumen.

Some embodiments of a laser ablation catheter may include a liquid filled waveguide having a catheter tube with an inner layer with a first index of refraction and an optical fluid disposed within and completely filling an inner lumen of the catheter tube. The optical fluid may have a second index of refraction which is greater than the first index of refraction. The liquid filled waveguide may further have a distal optical window in optical communication with the optical fluid and disposed in sealed relation to an inner surface of the catheter tube at a distal end of the catheter tube. A proximal optical window may further be in optical communication with the optical fluid and disposed in a liquid sealed relation to the inner surface of the catheter tube at a proximal end of the catheter tube. The laser ablation catheter may also have an outer jacket that is disposed over an outer surface of the catheter tube. A proximal end of the outer jacket may be disposed at a proximal portion of the catheter tube and a distal end of the outer jacket may be disposed at a distal end of the catheter tube. For some embodiments, the outer jacket may include a tubular configuration having an inner lumen with an inner surface, the inner surface of the inner lumen being configured to slide over an outer surface of the catheter tube with a close fit therebetween. The outer jacket may also have a tubular jacket body with a longitudinal stiffness greater than a proximal portion of the catheter tube. The tubular jacket body may have an inner jacket layer including a first material and an outer jacket layer including a second material. The tubular jacket body may also have a reinforcement that is crush resistant and disposed between the first layer and second layer of the tubular jacket body.

Certain embodiments are described further in the following description, examples, claims and drawings. These features of embodiments will become more apparent from the following detailed description when taken in conjunction with the accompanying exemplary drawings.

### BRIEF DESCRIPTION OF THE DRAWINGS

FIG. 1 is a perspective view of a laser system embodiment including a laser, a laser ablation catheter coupled to the laser and an optional support catheter disposed over the laser ablation catheter.

FIG. 2 is an elevation view in partial section of the laser ablation catheter embodiment of the laser system embodiment of FIG. 1, the laser ablation catheter including an outer jacket.

FIG. 3 is an elevation view in longitudinal section of a distal section of the laser ablation catheter embodiment of FIG. 2.

FIG. 4 is an enlarged view of the distal section of the laser ablation catheter embodiment of FIG. 3 indicated by the encircled portion 4-4 in FIG. 3.

FIG. 4A is an enlarged view of an embodiment of a distal section of the laser ablation catheter of FIG. 3 wherein a distal optical window thereof is crimped in place.

FIG. 4B is an enlarged view of an embodiment of a distal section of the laser ablation catheter of FIG. 3 wherein a distal optical window thereof is crimped in place along with a distal end of the catheter tube and a distal end of the outer jacket and the distal optical window includes a rounded edge.

FIG. 5 is a transverse cross section view of the laser catheter ablation embodiment of FIG. 2 taken across lines 5-5 of FIG. 2.

FIG. 6 is an enlarged view of the encircled portion of the laser ablation catheter embodiment of FIG. 5.

FIG. 7 is a transverse cross section view of the laser ablation catheter embodiment of FIG. 1 taken across lines 7-7 of FIG. 2.

FIG. 8 is a transverse cross section view of the laser ablation catheter embodiment of FIG. 1 taken across lines 8-8 of FIG. 1.

FIG. 9 is a transverse cross section view of the laser ablation catheter embodiment of FIG. 1 taken across lines 9-9 of FIG. 1.

FIG. 10 is a longitudinal section view of the laser ablation catheter embodiment of FIG. 9 taken across lines 10-10 of FIG. 9.

FIG. 10A is a longitudinal section view of another embodiment of the laser ablation catheter embodiment of FIG. 9 taken across lines 10-10 of FIG. 9.

FIG. 10B is a longitudinal section view of another embodiment of the laser ablation catheter embodiment of FIG. 9 taken across lines 10-10 of FIG. 9.

FIG. 10C is an enlarged view in partial longitudinal section of a proximal optical window embodiment disposed within a laser coupler embodiment disposed at the distal end of the laser ablation catheter embodiment of FIG. 1.

FIG. 11 is an elevation view of an outer jacket embodiment.

FIG. 12 is a transverse cross section view of the outer jacket embodiment of

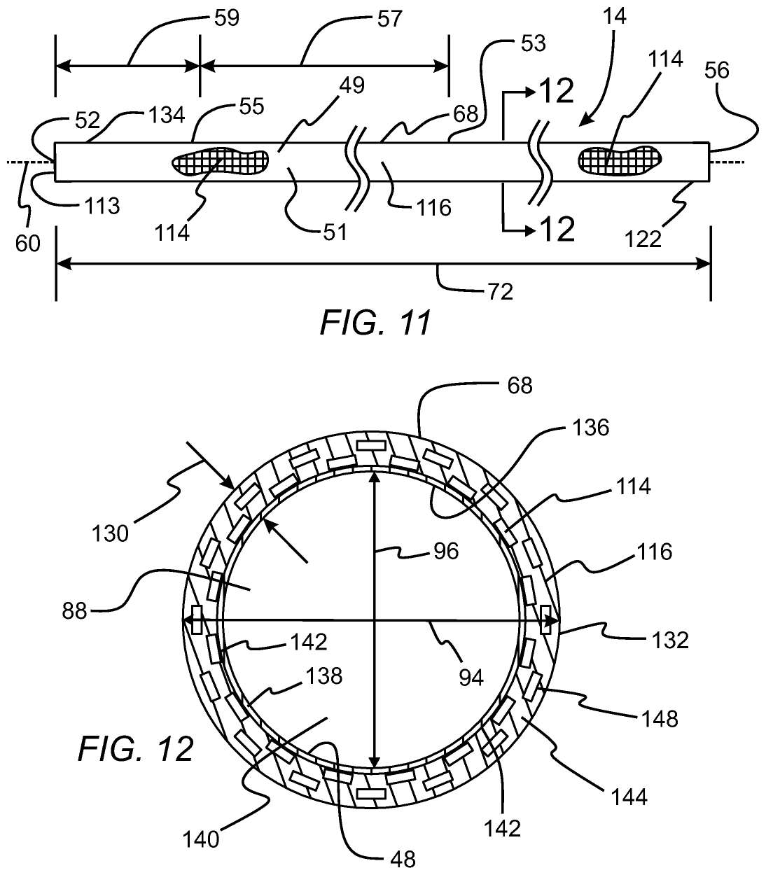

FIG. 11 taken along lines 12-12 of FIG. 11.

FIG. 13 is a perspective view of a section of a reinforcement embodiment configured as braided reinforcement ribbons and shown without additional structure of the corresponding laser ablation catheter for clarity of illustration.

FIG. 14 is a transverse cross section view of the reinforcement embodiment of FIG. 13 taken along lines 14-14 of FIG. 13.

FIG. 14A is an enlarged cross section view of the reinforcement of FIG. 14 taken across lines 14A-14A of FIG. 14.

FIG. 15 is a perspective view of a section of a reinforcement embodiment configured as a coiled reinforcement wire shown without additional structure of the corresponding laser ablation catheter for clarity of illustration.

FIG. 15A is a transverse cross section view of the reinforcement wire of FIG. 15 taken across lines 15A-15A of FIG. 15.

FIG. 16 is a perspective view of an axial section of a reinforcement embodiment configured as a coiled reinforcement ribbon shown without additional structure of the laser ablation catheter for clarity of illustration.

FIG. 16A is a transverse cross section view of the reinforcement ribbon of FIG. 16 taken across lines 16A-16A of FIG. 16.

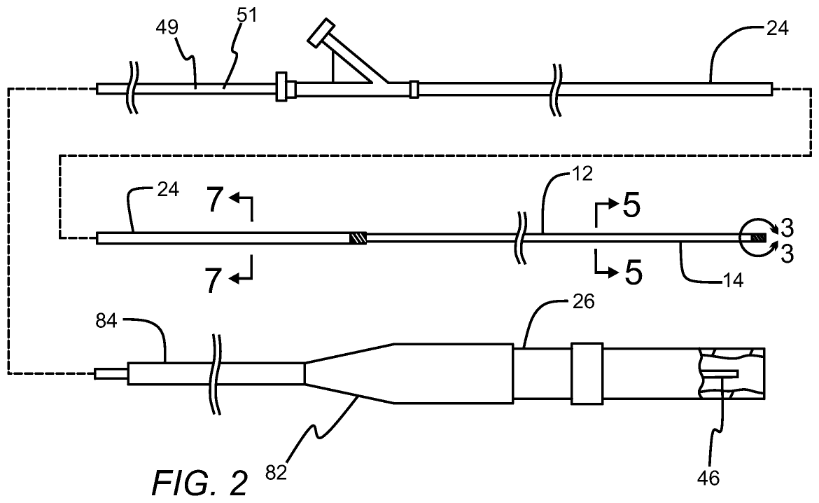

FIG. 17 is an elevation view of an embodiment of the laser ablation catheter embodiment of FIG. 2 that includes an eccentric guidewire lumen.

FIG. 18 is a transverse section view of the laser ablation catheter embodiment of FIG. 17 taken along lines 18-18 of FIG. 17.

FIG. 19 is a longitudinal section view of a distal section of the laser ablation catheter embodiment of FIG. 17 indicated by the encircled portion 19-19 of FIG. 17.

FIG. 20 is an elevation view in partial section of the laser ablation catheter embodiment of FIG. 2 in use ablating a blockage disposed within a body lumen.

FIG. 21 is an elevation view in partial section of the laser ablation catheter embodiment of FIG. 17 in use being guided by a guidewire and ablating a blockage disposed within a body lumen.

The drawings are intended to illustrate certain exemplary embodiments and are not limiting. For clarity and ease of illustration, the drawings may not be made to scale, and, in some instances, various aspects may be shown exaggerated or enlarged to facilitate an understanding of particular embodiments.

### DETAILED DESCRIPTION

Laser system embodiments discussed herein may generally include a laser which is configured to supply laser energy to a laser ablation catheter which may be operatively coupled to the laser. Such laser ablation catheter embodiments may be disposable in some cases and include a liquid core waveguide used to transmit the laser energy produced by the laser and coupled to the waveguide of the laser ablation catheter. Laser ablation catheters that include liquid core waveguides may sometimes have a low index of refraction coating which may be applied to an inner surface of a fluoropolymer catheter tube of the laser ablation catheter embodiments to yield high levels of transmission of laser energy through an optical fluid disposed within the catheter tube. In some cases, suitable laser energy for use with the laser ablation catheter embodiments discussed herein may have a wavelength in the ultraviolet range and in some particular cases may have a wavelength of about 308 nm. The laser energy may be pulsed as well.

Examples of laser system embodiments and associated laser ablation catheter embodiments are discussed in commonly owned U.S. Pat. No. 9,700,655, filed Oct. 12, 2012, by J. Laudenslager et al. and issued Jul. 11, 2017, titled “Small Flexible Liquid Core Catheter for Laser Ablation in Body Lumens and Methods for Use”, U.S. Patent Publication No. 2015/0105714, serial no. 14/515,435, filed Oct. 15, 2014, by J. Laudenslager et al., titled “Methods and Devices for Treatment of Stenosis of Arteriovenous Fistula Shunts”, now U.S. Pat. No. 9,962,527, issued May 8, 2018, U.S. Patent Publication No. 2017/0143424, serial no. 15/359,412, filed Nov. 22, 2016, by J. Laudenslager et al., titled “Laser Ablation Catheters Having Expanded Distal Tip Windows for Efficient Tissue Ablation,” now U.S. Pat. No. 10,555,772, issued Feb. 11, 2020, and U.S. Patent Publication No. 2019/0290356, serial no. 16/359,889, filed Mar. 20, 2019, by Z. Wood et al., titled “Liquid Filled Ablation Catheter with Overjacket,” each of which is incorporated by reference herein in its entirety.

Fluoropolymers such as fluorinated ethylene propylene (FEP), ethylene-tetrafluoroethylene-hexafluoropropylene (EFEP), ethylene tetrafluoroethylene (ETFE) and others may be desirable for use as catheter tubing material in these types of liquid core waveguide applications because catheter tubing formed from these materials may have a high UV transmission and low index or refraction amorphous Teflon AF® coatings may adhere well to such fluoropolymers. In addition, such fluoropolymer catheter tubes may be configured to be very flexible when axial loads are applied to laser ablation catheter embodiments that include catheter tubes made from such materials. As discussed above, catheter tubes which are formed from these materials may be coated with low index of refraction optical coatings in order to create an inner layer on the inner surface of the catheter tube. For some embodiments, the inner layer may have a first index of refraction and an optical fluid disposed within an inner lumen of such catheter tubes may have a second index of refraction which is greater than the first index of refraction resulting in internal reflection at the inner layer and thereby forming a waveguide with a tubular configuration which may be configured for transmission of high energy laser energy through the optical fluid.

In some cases, an optical coating in liquid form may be applied to the inner surface of the catheter tube, and then the optical coating may be thermally “cured” onto the inner surface via an appropriate thermal cycle thereby creating the inner layer. For some embodiments, this thermal cycle may be performed at elevated temperatures. In some instances, such elevated temperatures may possibly degrade and/or distort laser catheter components fabricated from non-fluoropolymer materials whereby physical distortion or softening of the materials may occur due to the high temperatures imposed on these materials. For this reason, it may be useful to perform such thermal cycle embodiments on the catheter tube and the optical coating prior to the addition of any other components of the corresponding laser ablation catheter which may include non-fluoropolymer polymer materials or metals of the laser ablation catheter which may be degraded by the thermal cycle.

Additionally, in some cases, the surface properties of the inner surface of a catheter tube which is formed from a fluoropolymer material may need to be altered in order to improve the adhesion of the optical coating to the inner surface. For example, the inner surface of the catheter tube may be chemically etched, plasma etched, or the like in order to obtain a desired level of adhesion between the catheter tube inner surface and the inner layer optical coating after the curing process.

As discussed above, a catheter tube which is formed from fluoropolymer material may exhibit suitable flexible behavior when subjected to axial loads, the flexibility of the catheter tube allowing for advancement of the respective laser ablation catheter through potential tortuous paths of body lumens of a patient or the like during a catheter procedure. In some cases, however, the flexibility of the catheter tube material may lead to distortion of the catheter tube which may in turn decrease the optical performance of the laser ablation catheter.

Distortion of the catheter tube may occur when a user of the laser ablation catheter applies an inward radial load to the catheter tube during manipulation of the catheter (e.g., fingers distorting/crushing an outside surface of the catheter tube while gripping the laser ablation catheter). In some other cases, distortion of the catheter tube may occur during advancement of the laser ablation catheter through a tortuous path, at which time an axial load is applied to the catheter tube. The axial load may lead to the distortion/kinking of the catheter tube due to insufficient longitudinal stiffness of the catheter tube during advancement through the tortuous path.

It may therefore be useful in some instances to implement devices and methods which increase the longitudinal stiffness and/or crush resistance of the catheter tube in order to avoid distortion of the catheter tube during a laser ablation catheter procedure. Currently some balloon catheters, fiber optic ablation catheters, and atherectomy catheters utilize guidewires which are operatively coupled to the respective catheter in order to increase the longitudinal stiffness and tractability of the catheter. Additionally, support catheters may be used to support these catheters.

For some laser ablation catheter embodiments that include a liquid core waveguide, a guidewire disposed through a central lumen of the catheter tube would significantly decrease the optical performance of the laser ablation catheter. The placement of an additional guidewire lumen in the center of the catheter tube may eliminate or greatly reduce the ability of the catheter to transmit laser energy by total internal reflection. For this reason, it may be useful for the longitudinal stiffness of the catheter tube to be optimized or improved through other means.

For some laser ablation catheter embodiments, the longitudinal stiffness of the catheter may be increased with the addition of an outer jacket which may be operatively coupled to the catheter tube waveguide portion of the laser ablation catheter. The outer jacket may be configured such that it slides over the outer surface of the catheter tube and includes a longitudinal stiffness which is greater than a longitudinal stiffness of the catheter tube in some cases. Such outer jacket embodiments may be configured to be resistant to radial loads (crush resistant) which are applied by a user of the laser ablation catheter. Because such outer jacket embodiments may be configured to slide over an outer surface of the catheter tube, the catheter tube and optical coating thereof may be exposed to an appropriate thermal cycle to form the inner layer and resulting optical waveguide properties prior to the attachment of the outer jacket. Thus, the outer jacket may include materials which would otherwise be degraded and/or distorted during application of the thermal cycle to the catheter tube and optical coating.

As discussed above, some laser ablation catheter embodiments discussed herein may be configured to allow for the optical coating and catheter tube to be thermally cycled and subsequently tested prior to the addition of an outer jacket. Thus, the material and/or materials of the outer jacket do not need to be subjected to the thermal cycle and can therefore be appropriately chosen and configured for their mechanical properties such that the outer jacket increases the longitudinal stiffness and crush resistance of the laser ablation catheter without degrading the properties of the finishes of the laser ablation catheter. This process also allows the material and/or materials of the outer jacket 14 to be selected in order to improve properties other than mechanical properties, such as radiation sterilization compatibility and the like.

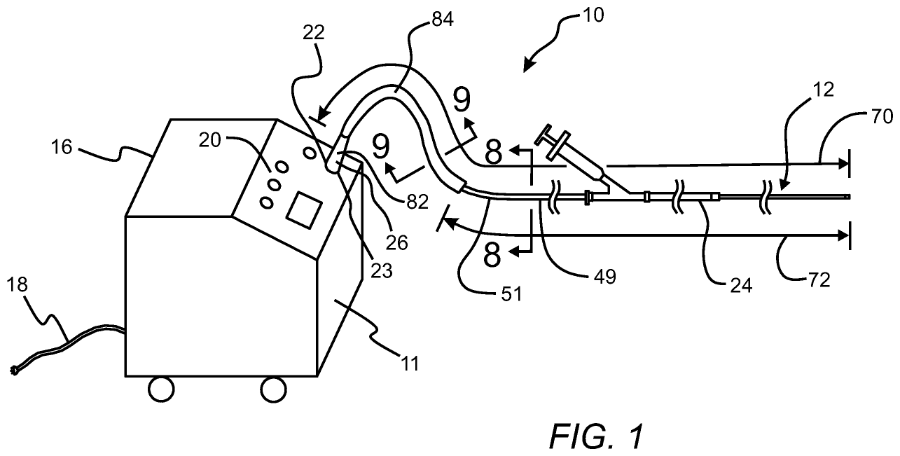

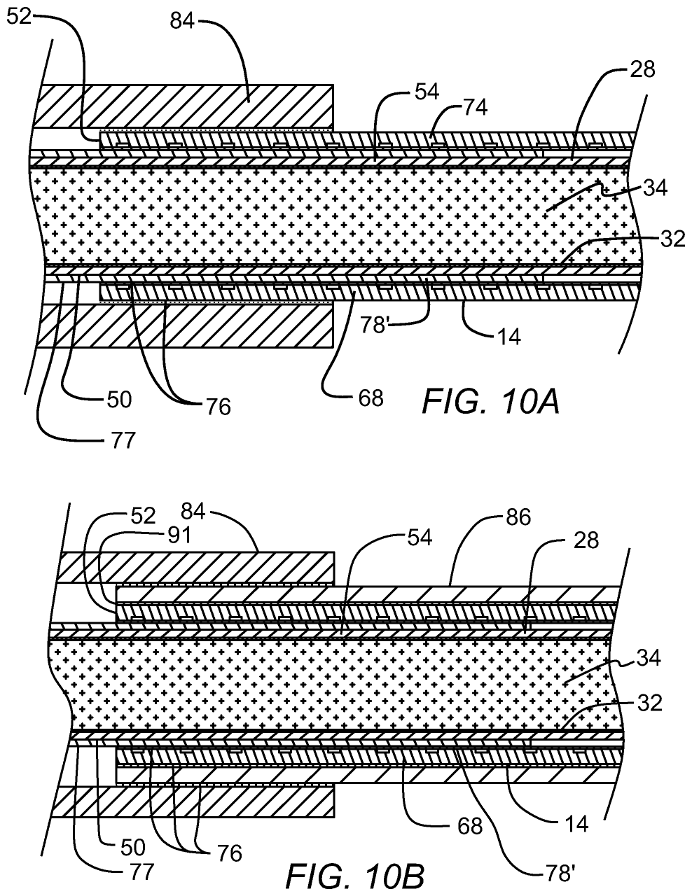

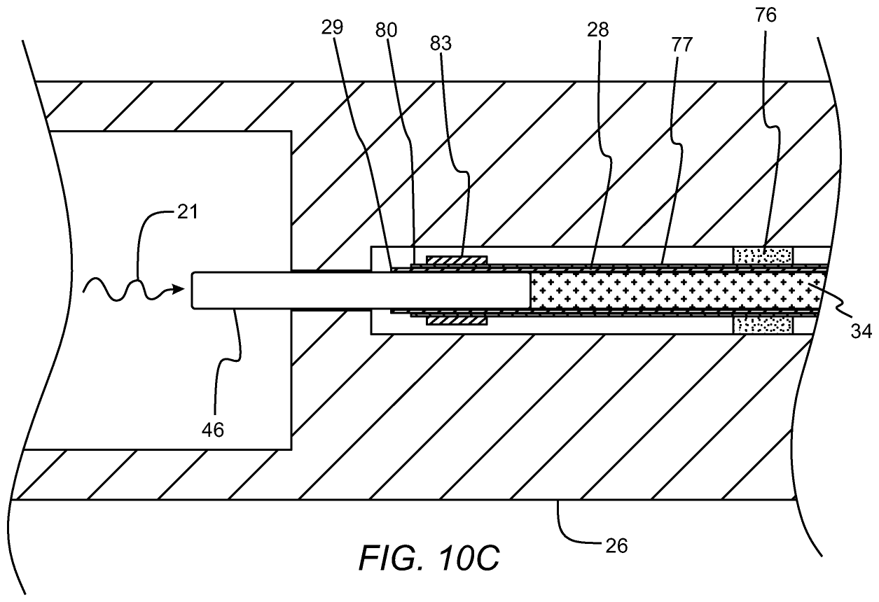

An embodiment of a laser system 10 including a laser ablation catheter 12 that may be used for tissue ablation, atherectomy as well as other indications is shown in FIGS. 1-3. The laser ablation catheter 12 may include a liquid filled waveguide and may also include an outer jacket 14. The laser system 10 may include a laser source 11, a housing 16, a power cord 18, a control panel 20 and an output coupler 22. For some embodiments, the laser system 10 may be configured to delivery laser energy 21 into the laser ablation catheter 12 as shown in FIG. 10C. In some cases, the laser system 10 may be configured to generate and deliver high energy and high power UV laser pulses with a wavelength of about 308 nm. For some embodiments, the high energy and high power UV laser energy output 21 of the laser system 10 may be pulsed and have sufficient energy fluence in order to allow for tissue ablation by laser energy 21 output from the laser ablation catheter 12. In some cases, the duration of each laser energy pulse from the laser system may be from about 10 nanoseconds to about 150 nanoseconds.

The laser ablation catheter 12 may include a laser coupler 26 which is disposed at a proximal end 23 of the laser ablation catheter 12 and which is configured to be operatively coupled to the output coupler 22 of the laser system 10. The laser coupler 26 may be configured to reliably position a laser energy input of the laser ablation catheter 12 relative to the output coupler 22 in order to facilitate an effective coupling of the laser energy 21 generated by the laser source 11 into the laser ablation catheter 12 as shown in FIG. 10C.

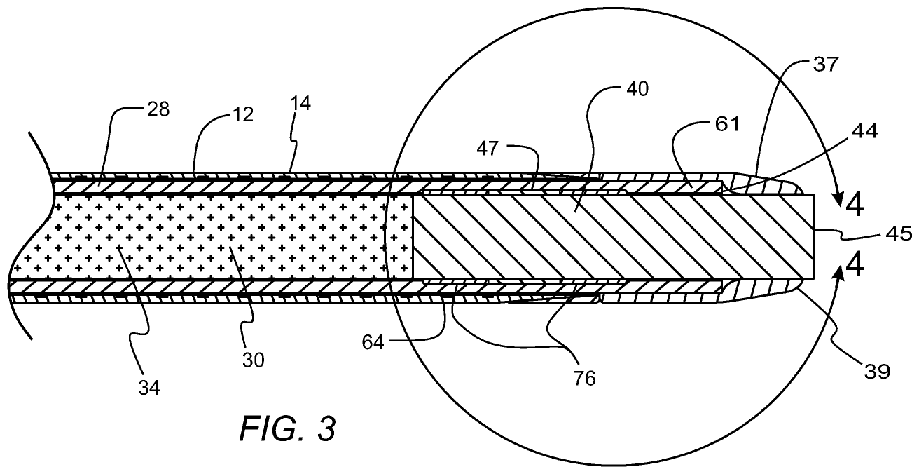

In some cases, the laser system 10 may optionally include a support catheter 24 which may have an inner lumen that is configured to readily slide over an outer surface of the laser ablation catheter 12. The laser ablation catheter 12 may thus be configured to readily slide axially back and forth within the inner lumen of the support catheter 24 as well. In some cases, the support catheter 24 may be configured to provide support and guidance to the laser ablation catheter 12 during a laser ablation procedure as shown in FIG. 20. The laser ablation catheter 12 may also include a liquid filled waveguide which is configured for the propagation of high powered laser energy 21 through the laser ablation catheter 12 for the purposes of ablating blockages 25 within body lumens 27 of vessels 31, such as arteries, and optionally performing atherectomy in a mammalian body such as a human body as well as other indications. Referring to FIGS. 3 and 5-6, such a liquid core waveguide may include a catheter tube 28 having a tubular configuration, an inner lumen 30 and an inner layer 32 which is disposed on an inner surface 33 of the inner lumen 30. In some embodiments, the material of the inner layer 32 may include an optical coating having a first index of refraction.

The liquid filled waveguide may also include a biocompatible ultraviolet transparent optical fluid 34 which is disposed within and which completely fills the inner lumen 30 of the catheter tube 28. The optical fluid 34 may have a second index of refraction which is greater than the first index of refraction of the inner layer 32. The different indices of refraction of the materials at an optical boundary 36 disposed between the inner layer 32 and the optical fluid 34 may be configured to allow for total internal reflection at the optical boundary 36 thereby forming an optical waveguide and permitting propagation of high powered laser energy through the waveguide. It may be useful for suitable optical fluid embodiments to efficiently transmit high power laser energy having a wavelength in the UV spectrum and particularly laser energy at wavelengths of about 308 nm with low optical losses and without degradation of the optical fluid embodiment 34. Embodiments of suitable optical fluids 34 may include materials such as purified water, purified saline solutions and the like.

In some instances, the quality and/or efficiency of optical transmission through the waveguide may be dependent upon the surface quality of the optical boundary 36 as shown in FIG. 6. In particular, a smooth continuous optical boundary 36 which conforms to the inner surface 33 of the inner lumen 30 of the catheter tube 28 will generally outperform the optical transmission of a rougher more discontinuous optical boundary 36 due to optical scattering losses etc. For this reason, it may be desirable to maximize the smoothness and continuity of an inner surface 38 of the inner layer 32 which along with the optical fluid 34 forms the optical boundary 36.

The laser ablation catheter 12 may also include an ultraviolet grade elongated distal optical window 40 as shown in FIG. 3 which may be disposed in liquid sealed relation to the inner surface 33 (see FIG. 6) of the catheter tube 12 at a distal end 44 of the catheter tube 28. The distal optical window 40 may be in contact and optical communication with the optical fluid 34 which is disposed within the inner lumen 30 of the catheter tube 28 thereby allowing for the efficient transmission of laser energy from the optical fluid 34 through the distal optical window 40. For the embodiment shown, the input surface of the distal optical window 40 is in direct fluid contact with the optical fluid 34 for efficient transmission of light energy through the transition between those elements.

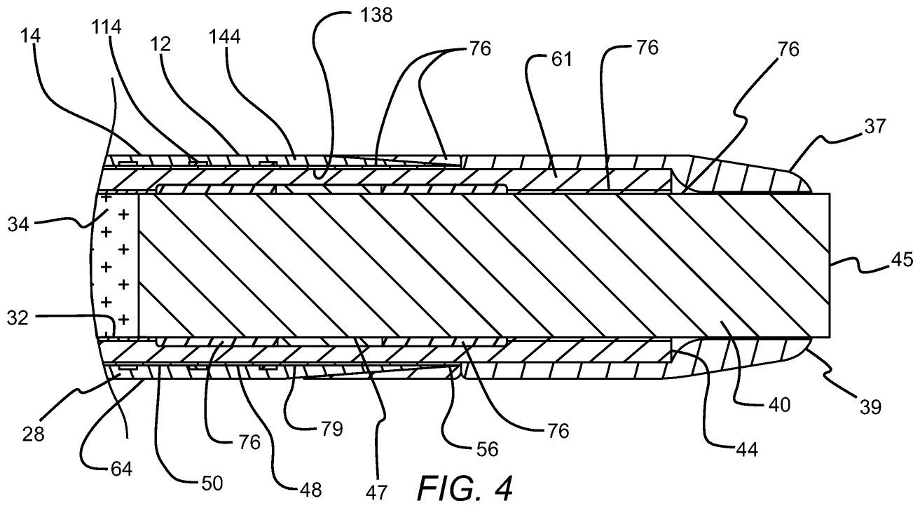

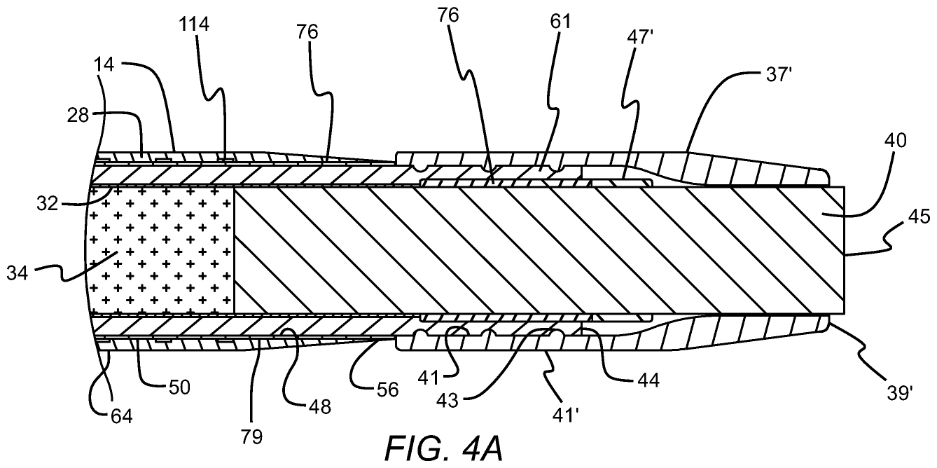

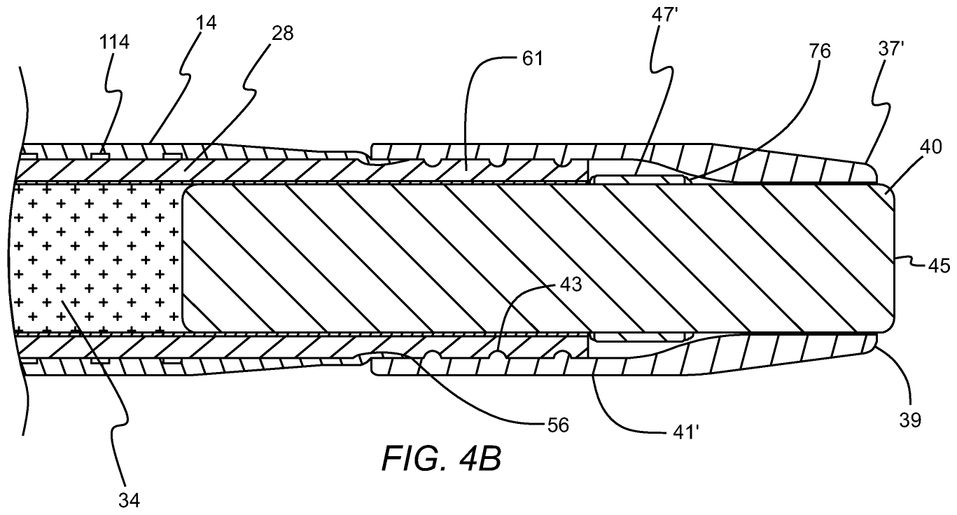

In some cases, the distal optical window 40 may be polished by conventional methods such as lapping or the like which may leave a square shaped edge disposed about the perimeter of the output surface as illustrated on the distal optical window embodiment 40 shown in FIG. 4. In other cases, distal optical window embodiments 40 may have a surface or surfaces polished by other methods such as laser polishing leaving a laser polished surface on the output surface of the distal optical window, input surface of the distal optical window 40 or on both the input and output surfaces of the distal optical window 40. Such laser polishing methods may be performed so as to leave a rounded or chamfered edge disposed about the perimeter of the output surface or any other surface which has been so polished. The distal optical window embodiment 40 shown in FIG. 4B includes both an input surface and output surface which have been laser polished so as to leave a rounded and chamfered edge disposed about each of those respective surfaces. Any of the distal optical windows 40 or proximal optical windows 46 (discussed below) may have such laser polished surfaces with rounded edges disposed thereabout. The chamfered or rounded edge or edges of the optical windows 40, 46 may be useful during assembly of some laser catheter embodiments 12 in order to facilitate insertion the optical window 40, 46 into the inner lumen of the coated catheter tube 28 without scratching or peeling the inner surface or coating thereof.

For some embodiments the distal optical window 40 may be disposed within embodiments of a distal housing 37 which may have a generally tubular configuration. The distal housing 37 may be secured to both the distal optical window 40 and to the catheter tube 28 by any suitable means such as adhesive bonding and may be formed from any suitable material including polymers and metals. Some distal housing embodiments, including distal housing embodiment 37′ discussed below, may be formed from a metal such as stainless steel, titanium or the like, and such embodiments may be secured to the distal optical window by crimping the distal housing 37′ to the distal optical window 40 and to the catheter tube 28. The distal housing 37′ when made from such metals may also serve as a radiopaque marker in some cases.

The laser ablation catheter 12 may also include a proximal optical window 46 (see FIG. 2) which may have a cylindrical configuration and which may be disposed within the laser coupler 26, with the proximal optical window 46 being disposed in a liquid sealed relation to the inner surface 33 (see FIG. 6) of the catheter tube 28. The proximal optical window 46 may be in optical communication and direct contact with the optical fluid 34 disposed within the inner lumen 30 of the catheter tube 28, thereby allowing for the efficient optical transmission of laser energy 21 from the laser source 11, through the proximal optical window 46, and into the optical fluid 34. The laser energy 21 may then be propagated through the liquid core waveguide (formed by the catheter tube 28, the inner layer 32, and the optical fluid 34), through the distal optical window 40, and thereafter be emitted from an output surface 45 of the distal optical window 40 and into a blockage 25 disposed within a human body or for any other suitable indication.

In some instances, the materials which form the respective components of the waveguide (including the catheter tube 28, the inner layer 32, and the optical fluid 34) may be chosen such that each material is not degraded by a thermal cycle which the waveguide may be subjected to during the formation of the inner layer 32 as discussed above. For example, the catheter tube 28 may be formed from FEP, a material which will easily endure a 204 degrees centigrade (for about a 120 minute duration in some cases) thermal cycle that may be useful to the “cure” an inner layer 32 which may in turn be formed from an amorphous Teflon AF®/Fluorinert FC-40® solution or such a solution using any other suitable solvent in place of the Fluorinert FC-40 solvent as well. However, in some cases and for some indications, catheter tubes 12 formed from thermally suitable materials such as FEP may lack the longitudinal stiffness and crush resistance which are desirable in order to minimize/avoid distortion/optical damage to the waveguide during a procedure using the laser ablation catheter 12. In some cases, the catheter tube 28 may also be made from or include fluoropolymers such as ETFE. For some embodiments, the addition of an outer jacket 14 to the catheter tube 28 of the laser ablation catheter 12 may allow for the separate processing of some waveguide components (including the catheter tube 28 and inner layer 32) and add longitudinal stiffness and crush resistance to the structure of the laser ablation catheter.

Referring to FIGS. 8-10, an inner surface 48 of the outer jacket embodiment 14 may be disposed over and secured relative to an outer surface 50 of the catheter tube 28. The outer jacket 14 has a proximal end 52 that may be disposed at a proximal portion 54 or disposed adjacent a proximal end 29 of the catheter tube 28. A distal end 56 of the outer jacket may be disposed adjacent or just proximal of the distal end 44 of the catheter tube 28 (as shown in FIG. 4). The outer jacket 14 may be secured relative to the catheter tube 28 such that the outer jacket 14 remains substantially fixed along a longitudinal axis 60 (see FIG. 11) thereof relative to the catheter tube 28.

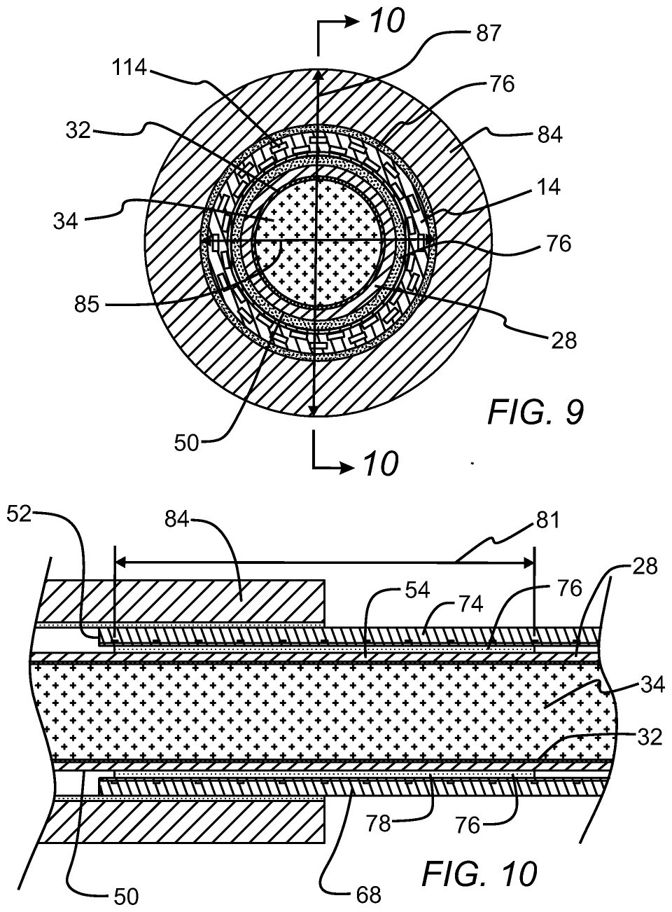

In some cases, the catheter tube 28 may have an axial length 70 (see FIG. 1) of about 200 cm to about 260 cm. The outer jacket 14 may have a similar axial length 72 (see FIG. 11) of about 200 cm to about 260 cm. A proximal portion 74 of the outer jacket 14 may be adhesively bonded to the catheter tube 28 (see FIGS. 9 and 10) by applying an adhesive 76 between the inner surface 48 of the outer jacket 14 and the outer surface 50 of the catheter tube 28 thereby forming a proximal bond section 78. The proximal portion 74 of the outer jacket 14 may also be crimped to the catheter tube 28 and/or the proximal optical window 46 in some cases.

With regard to the interface between the catheter tube 28 and outer jacket 14 with the laser coupler 26, and referring to FIG. 10A, some laser ablation catheter embodiments 12 may include a proximal inner sleeve 77. The proximal inner sleeve 77 may have a tubular configuration that is disposed over the catheter tube 28 with a distal section of the proximal inner sleeve 77 disposed within the inner lumen 88 of the outer jacket 14 and axially overlapping the proximal end 52 of the outer jacket 14. For some embodiments, the proximal end 80 of the proximal inner sleeve 77 may extend proximally to the proximal optical window 46 and be axially coextensive or substantially axially coextensive with the proximal end 29 of the catheter tube 28 disposed within the laser coupler 26 as shown in FIG. 10C. As such, the proximal end 80 of the proximal inner sleeve 77 may be crimped or otherwise secured to the proximal optical window 46 as well with a crimp ring 83 in the same manner as the proximal end 29 of the catheter tube 28. In addition, a proximal bond section 78′ may be formed between the proximal portion 74 of the outer jacket 14 and a distal section of the proximal inner sleeve 77 with the use of a suitable adhesive 76 between relative surfaces thereof as shown in FIG. 10A. Suitable adhesives may include cyanoacrylates, epoxies as well as U.V. cured adhesives and the like. The proximal inner sleeve 77 may include a thin walled tubular structure made from a material having high strength and lubricious outer surface such as a PTFE/polyimide hybrid material or the like. In some cases, the proximal inner sleeve 77 may have an axial length of about 1 inch to about 6 inches.

Referring to FIG. 10B, an optional outer jacket support 86 is shown that may be configured to provide additional strength and support to a proximal section of the outer jacket 14 and proximal section of the laser ablation catheter 12 generally. For some embodiments, the outer jacket support 86 may have a tubular configuration with an inner lumen 91 disposed over an outer surface 15 of the outer jacket 14. The outer jacket support 86 may generally extend distally from a proximal section or proximal end 52 of the outer jacket 14 to an axial position about 30 cm to about 50 cm proximal of the distal end 56 of the outer jacket 14. In some cases, the outer jacket support 86 may include a wall thickness of about 0.001 inches to about 0.003 inches. For some embodiments, the outer jacket support 86 may include polymers such as polyurethane, polyether block amide, polyimide or hybrids such as PTFE/polyimide hybrid materials. The outer jacket support 86 may also include materials such as chlorinated fluoropolymers such as poly (chloro trifluoro ethylene) (PCTFE) as well as other fluoropolymers including ethylene tetrafluoro ethylene (ETFE) and EFEP that may be suitable for adhesive bonding and resistant to certain types of sterilization such as radiation sterilization.

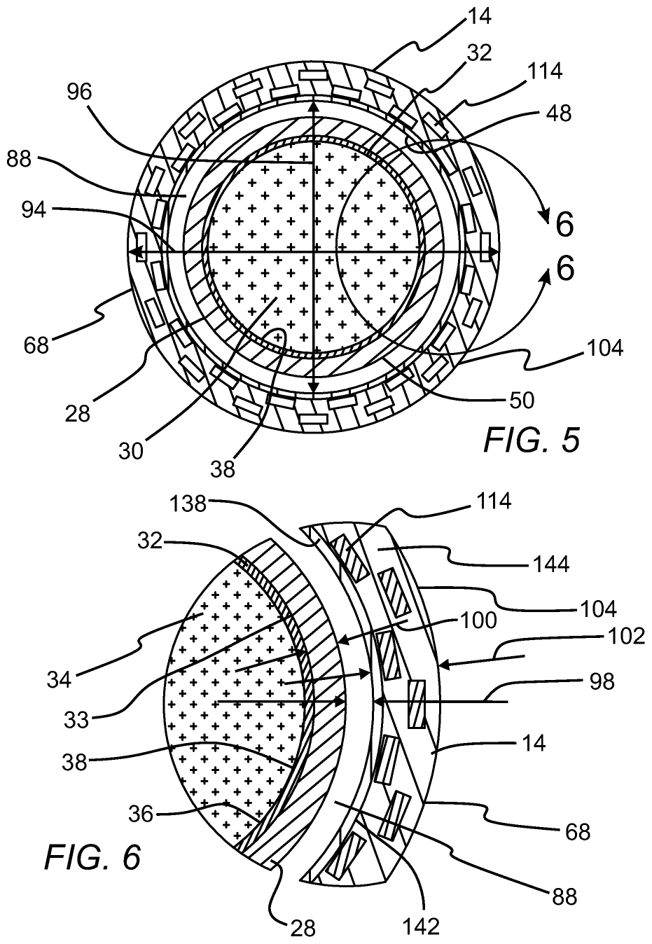

As shown in FIGS. 3 and 4, the distal portion 64 of the outer jacket 14 may be adhesively bonded to the catheter tube 28 by applying an adhesive 76 between the inner surface 48 of the outer jacket 14 and the outer surface 50 of the catheter tube 28 thereby forming a distal bond section 79. Any suitable adhesive such as a cyanoacrylate, epoxy, ultraviolet (U.V.) cured adhesive or the like may be used to create the proximal bond section 78 and/or the distal bond section 79. For some embodiments, an axial length 81 of the proximal bond section 78 may be about 0.039 inches to about 3 inches (see FIG. 10). For the axial section disposed between the proximal bond section 78 and the distal bond section 79 the outer jacket 14 may be unbonded to the catheter tube 28 (see FIG. 5).

In some cases, the distal end 44 or distal portion 61 of the catheter tube 28 may be secured to the distal optical window 40, or an outer surface thereof, with an adhesive bond and with a distal end 44 of the catheter tube 28 disposed proximally of the distal end 45 of the distal optical window 40 as shown in FIGS. 3 and 4. For such an embodiment, the distal end 44 of the catheter tube 28 may be disposed about 1 mm to about 2 mm proximally from the distal end 45 of the distal optical window 40. In addition, a distal end 56 of the outer jacket 14 may be secured to an outside surface of the distal portion 61 of the catheter tube 28 with the distal end 56 of the outer jacket 14 disposed proximally of the distal end 44 of the catheter tube 28. In some cases, the distal end 56 of the outer jacket 14 may be disposed about 1 mm to about 2 mm proximally of the distal end 44 of the catheter tube 28. For such embodiments, an outer surface of the outer jacket 14 may taper distally to a reduced outer transverse dimension at the distal end 56 and/or distal portion 64 of the outer jacket 14. A radiopaque marker band 47 may be disposed between the inside surface of the catheter tube 28 and an outside surface of the distal optical window 40. For some embodiments, the radiopaque marker band 47 may optionally be disposed on an outside surface of the distal optical window 40 distal of the distal end 44 of the catheter tube 28 and within an inside surface a distal housing 37 as shown in the embodiment of FIG. 4A.

Still referring to FIG. 4, for such an embodiment of the laser ablation catheter 12, the distal housing 37 may be secured to an outside surface of the distal optical window 40 and an outer surface of the distal end 44 of the catheter tube 28. Embodiments of the distal housing 37 may have a rounded bullet shaped outer surface contour and a stepped inner lumen having an inner surface configured to have a close fit with an outside surface adjacent the distal end 45 of the distal optical window 40 and an outer surface of the distal end 44 and/or distal portion 61 of the catheter tube 28. For some embodiments, the distal housing may be secured to the distal optical window 40 and catheter tube 28 with a distal end 39 of the distal housing 37 disposed proximally of the distal end 45 of the distal optical window 40. For some embodiments, the distal end 39 of the distal housing 37 may be disposed about 0.05 mm to about 0.5 mm proximally of the distal end 45 of the distal optical window 40. In some cases, the distal housing 37 may include a polymer material including nylon, polyether block amides including Pebax®, polyurethanes including Pellethane®, fluoropolymers or the like. For some embodiments, the material of the distal housing may have a hardness of about 70 Shore D to about 74 Shore D. In some instances, the polymer of the distal housing 37 may include a radiopaque material including powdered materials such as bismuth, barium, tantalum or the like. The distal housing 37, may in some cases also include metals such as stainless steel, nickel titanium alloy, titanium or the like.

FIG. 4A illustrates another embodiment of a distal section of an embodiment of the laser ablation catheter 12 which includes a distal housing 37′ which is secured to an outer surface of the distal end 44 and/or distal portion 61 of the catheter tube 28 with a crimped joint. For the embodiment shown, the distal housing 37′ may include a crimpable metal material, including stainless steel, titanium, nickel titanium alloys or the like. The distal housing embodiment 37′ may also have a rounded bullet shaped outer surface and a stepped inner lumen with an inner surface configured to have a close fit with an outside surface of the distal end 45 and/or distal portion of the distal optical window 40 and an outer surface of the distal end 44 and/or distal portion 61 of the catheter tube 28 prior to crimping. A proximal section 41′ of the stepped inner lumen of the distal housing 37′ may be configured to crimp to the distal section 61 of the catheter tube 28 to generate the crimp joint therebetween and may further include a plurality of crimp rings 43 disposed on an inner surface thereof. In some cases, the crimp rings 43 may include circumferential annular rings extending inwardly from the inner surface of the proximal section 41′ of the stepped inner lumen of the distal housing 37′.

For such embodiments, the distal housing 37′ may be secured to the catheter tube 28 with a distal end 39′ of the distal housing 37′ disposed proximally of the distal end 45 of the distal optical window 40. In some cases, the distal end 39′ of the distal housing 37′ may be disposed about 0.05mm to about 0.5 mm proximally of the distal end 45 of the distal optical window 40. In some cases, the catheter tube 28 may be secured to the distal optical window 40 with the distal end 44 of the catheter tube 28 disposed proximally of the distal end 45 of the distal optical window 40. In some instances, the distal end 44 of the catheter tube 28 is disposed about 2 mm to about 3 mm proximally from the distal end 45 of the distal optical window 40.

In some cases, the distal end 56 of the outer jacket 14 may be secured to the outside surface of a distal portion 61 of the catheter tube 28 with the distal end 56 of the outer jacket 14 disposed proximally of the distal end 44 of the catheter tube 28. Some embodiments may have the distal end 56 of the outer jacket 14 disposed under an inside surface of the proximal stepped section 41′ and crimped in place as shown in the embodiment of FIG. 4B. FIG. 4B shows proximal section 41′ of the stepped inner lumen of the distal housing 37′ having a configuration which is suitable to crimp to the distal section 61 of the catheter tube 28 and a distal end 56 of the outer jacket 14 into the crimp joint therebetween as shown. All other features, dimensions and materials of the crimp joint shown in the embodiment of FIG. 4B may be the same as or similar to those shown in the crimp joint embodiment of FIG. 4A. The distal optical window 40 shown in FIG. 4B also includes a laser polished input surface and a laser polished output surface which each have a rounded and/or chamfered edge disposed thereabout which may be useful to facilitate assembly of the distal section of the laser ablation catheter 12. The proximal optical window 46 may also have a laser polished input surface and a laser polished output surface which each have a rounded and/or chamfered edge disposed thereabout as shown in FIG. 10C which may be useful to facilitate assembly of the proximal section of the laser ablation catheter 12 as discussed above.

In some instances, the distal end 56 of the outer jacket 14 may be disposed about 1 mm to about 2 mm proximally of the distal end 44 of the catheter tube 28. For some embodiments, the outer jacket 14 may be secured to the catheter tube 28 with an adhesive bond. For some embodiments, the outer surface of the outer jacket 14 may be configured to taper distally to a reduced outer transverse dimension at the distal end 56 of the outer jacket 14. For some embodiments, a radiopaque marker band 47′ may be disposed on an outside surface of the distal optical window 40 and inside the stepped inner lumen of the proximal section 41′ of the distal housing 37′ as shown in the embodiment of FIG. B. In some cases, a proximal end of the radiopaque marker band 47′ may be disposed adjacent the distal end 44 of the catheter tube 28. For some embodiments, the crimpable metal of the distal housing 37′ may include titanium, stainless steel or any other suitable crimpable metal or alloy.

Regarding the construction of certain embodiments of the outer jacket 14, some outer jacket embodiments 14 which are discussed herein may include a manipulation section 49 (see FIGS. 1 and 2) which may include an axial section 51 of the outer jacket 14 which is disposed between the proximal portion 74 (see FIG. 10) of the outer jacket 14 and a central portion 53 of the outer jacket 14 (see FIG. 11). In some cases, the manipulation section 49 may represent the section of the outer jacket 14 which is most frequently manipulated/grasped by a user of the laser ablation catheter 12 while positioning and/or advancing the laser ablation catheter 12 during an ablation procedure (or any other suitable indication). In some cases, the manipulation section 49 may serve as a manual interface between the user and the laser ablation catheter 12. The manipulation section 49 may be configured to be crush resistant, kink resistant, and buckle resistant and may also be configured to have a longitudinal stiffness which is greater than the longitudinal stiffness of the respective portion of catheter tube 28 which is disposed within the manipulation section 49 of the outer jacket 14.

For some embodiments, the manipulation section 49 may have a proximal terminus 55 (see FIG. 11) which is an axial distance 59 of about 5 cm to about 50 cm distal of the proximal end 52 of the outer jacket 14, with the manipulation section 49 disposed distal to the proximal terminus 55. The manipulation section 49 may also have a proximal terminus that is axially coextensive with the distal end of the jacket strain relief 84. In some cases, the manipulation section may have an axial length 57 (see FIG. 11) of about 50 cm to about 150 cm. Any of the laser ablation catheter embodiments 12, 12′ discussed herein may include a manipulation section thereof having any suitable feature, dimension or material of the manipulation section 49 of the outer jacket embodiments 14 discussed above.

The laser ablation catheter 12 may also include a connector strain relief 82, and a jacket strain relief 84 (see FIGS. 1-2 and 9-10). Connector strain relief embodiments 82 may be operatively coupled between the laser coupler 26 and jacket strain relief embodiments 84 and may be configured to protect the catheter tube 28 and waveguide structure generally as well as the outer jacket 14 from traumatic bending due to high axial loads. The connector strain relief 82 may have a straight walled portion with a substantially constant outer diameter or outer transverse dimension as well as a tapered section which extends distally from the straight walled section tapering distally to a reduced outer transverse dimension. In turn the jacket strain 84 relief may be operatively coupled between the connector strain relief 82 and the outer jacket 14 and may be configured to protect the proximal portion 54 of the catheter tube 28 from bending damage due to high axial loads. A proximal end of the jacket strain relief 84 may be secured to a distal end of the connector strain relief 82 for some embodiments. The jacket strain relief 84 may be suitably secured to the connector strain relief 82 such that the jacket strain relief 84 remains fixed with respect to the connector strain relief 82. For example, the jacket strain relief 84 may be adhesively bonded to the connector strain relief 82 with a suitable adhesive such as cyanoacrylate, epoxy, U.V. cured adhesives or the like. For other embodiments, the connector strain relief 82 and jacket strain relief 84 may include a monolithic unitary single piece structure that may be molded together from a continuous material. The jacket strain relief 84 may also optionally include a tapered configuration which tapers distally to a reduced outer transverse dimension or diameter over any suitable axial section thereof, including from the junction with the connector strain relief 82 to a distal end of the jacket strain relief 84. Either of the respective tapered sections of the connector strain relief 82 or jacket strain relief 84 may be sized and configured to seal to a compatible inside contour of an open end of a fluid filled packaging tube (not shown). In some cases, laser ablation catheter embodiments 12, 12′ discussed herein may be disposed within the inner lumen of such packaging tubes during storage and shipment in order to increase the vapor pressure around the laser ablation catheter 12, 12′ and minimize or prevent a loss of optical fluid 34. Such a fluid tight seal between the tapered section of the connector strain relief 82 or tapered section of the jacket strain relief 84 may be useful in order to prevent the fluid disposed within the fluid filled packaging tube from escaping during storage and shipment.

For some embodiments, the jacket strain relief 84 may be monolithically formed from a suitable resilient polymer material such as nylon, polyether block amides including Pebax®, polyurethanes including Pellathane®, silicone rubber, or the like. The jacket strain relief 84 may also be made from materials such as fluoropolymers including PCTFE, ETFE or the like which may be particularly resistant to certain sterilization methods such as radiation sterilization. Alternatively, the jacket strain relief 84 may be formed from multiple layers of materials including nylon and Pebax®. For some embodiments the jacket strain relief 84 may have an inner diameter 85 (see FIG. 9) of about 0.002 inches to about 0.004 inches larger than the outer diameter 92 (see FIG. 8) of the catheter tube 28, and an outer diameter 87 of about 0.006 inches to about 0.050 inches larger than the outer diameter 92 of the catheter tube 28. Some jacket strain relief embodiments 84 may have an axial length of about 5 inches to about 20 inches. Other embodiments may include a jacket strain relief 84 having an axial length of about 2 inches to about 10 inches.

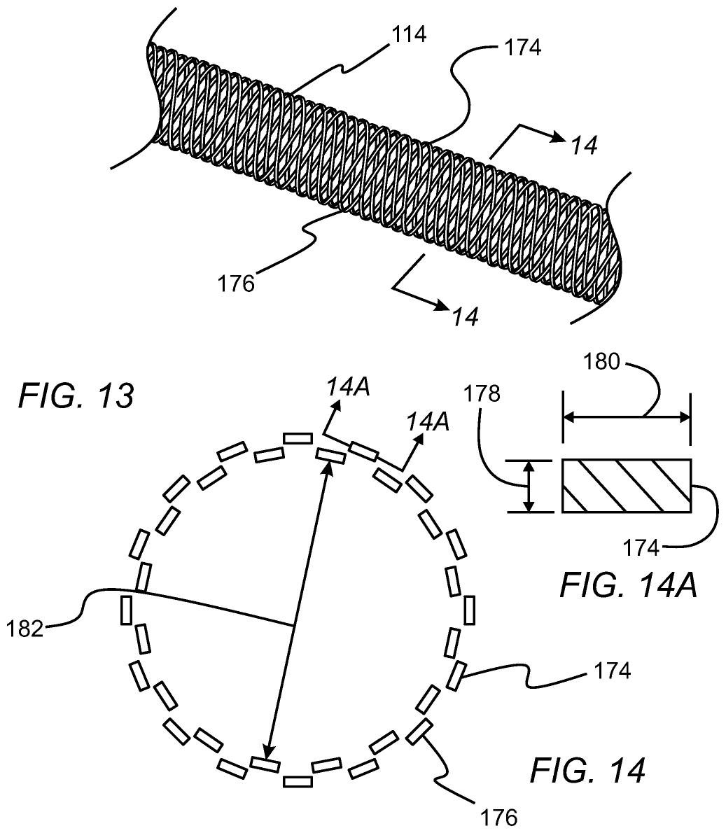

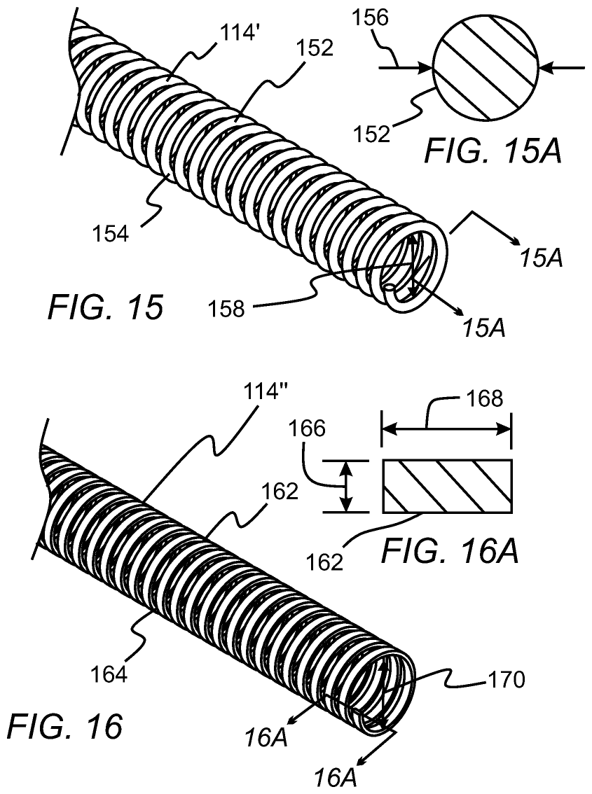

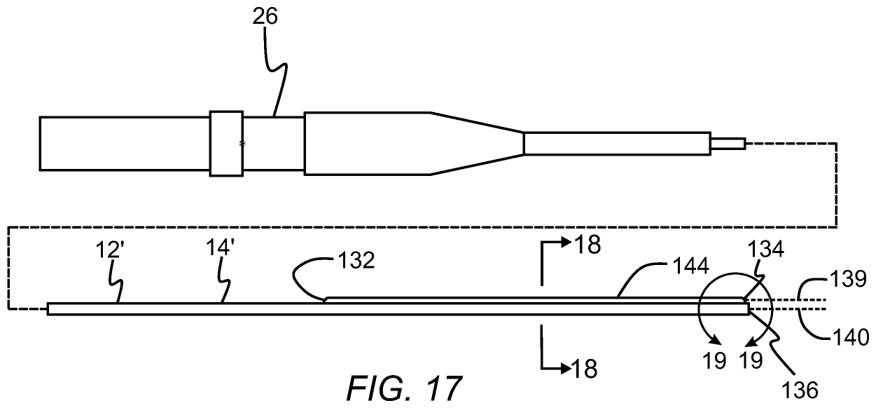

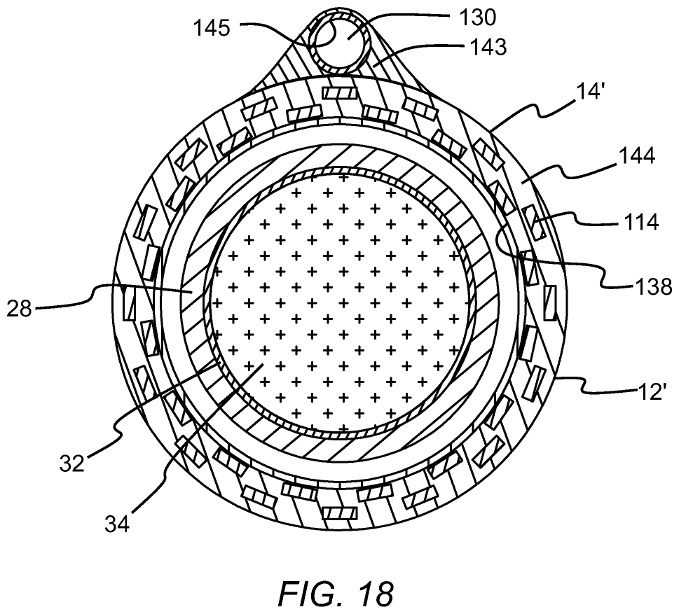

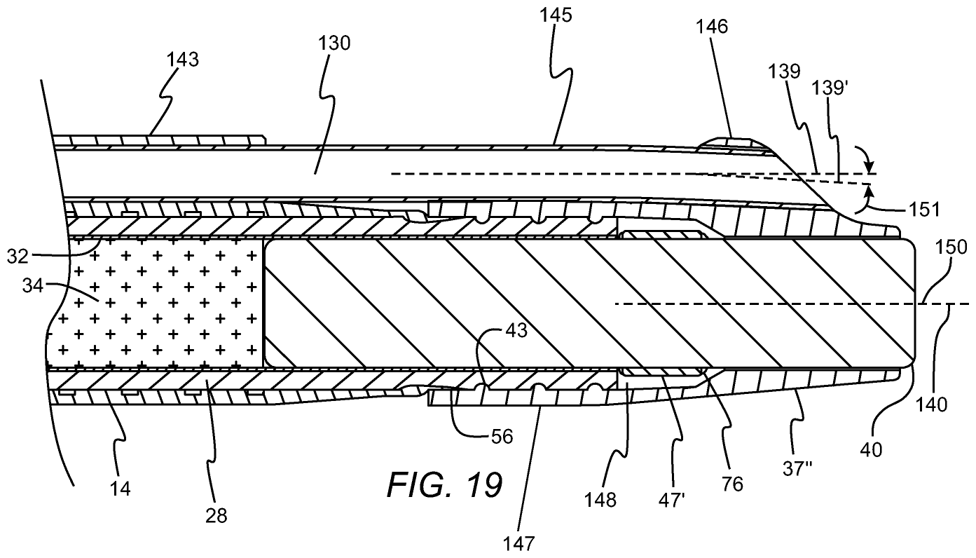

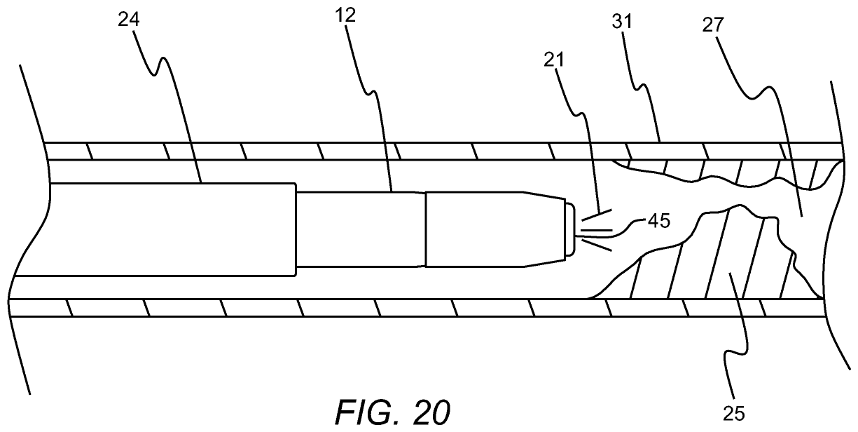

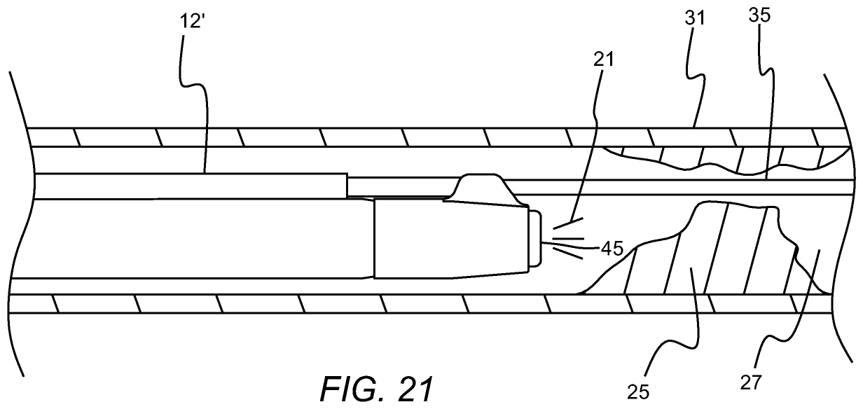

In some cases, the outer jacket 14 may have a tubular configuration and may include an inner lumen 88 that has an inner surface 48. As discussed above, the inner lumen 88 (see FIGS. 6 and 12) of the outer jacket 14 may be configured to slide over the outer surface 50 of the catheter tube 28 with a close fit between the inner surface 48 of the outer jacket 14 and the outer surface 50 of the catheter tube 28. In some cases, embodiments of the catheter tube 28 may have an inner diameter 90 (diameter of inner surface, see FIG. 8) of about 0.042 inches to about 0.044 inches and an outer diameter 92 (diameter of outer surface) of about 0.052 inches to about 0.054 inches. A respective outer jacket 14 may have an inner diameter 96 (diameter of inner surface, see FIG. 12) of about 0.0545 inches to about 0.0565 inches and an outer diameter 94 (diameter of outer surface) of about 0.064 inches to about 0.0665 inches. In some instances, for laser ablation catheter embodiments 12 discussed herein, the close fit between the outer surface 50 of the catheter tube 28 and the inner surface 48 of the inner lumen 88 of the outer jacket 14 may include a dimensional clearance 98 (see FIG. 6) of about 0.0005 inches to about 0.004 inches.

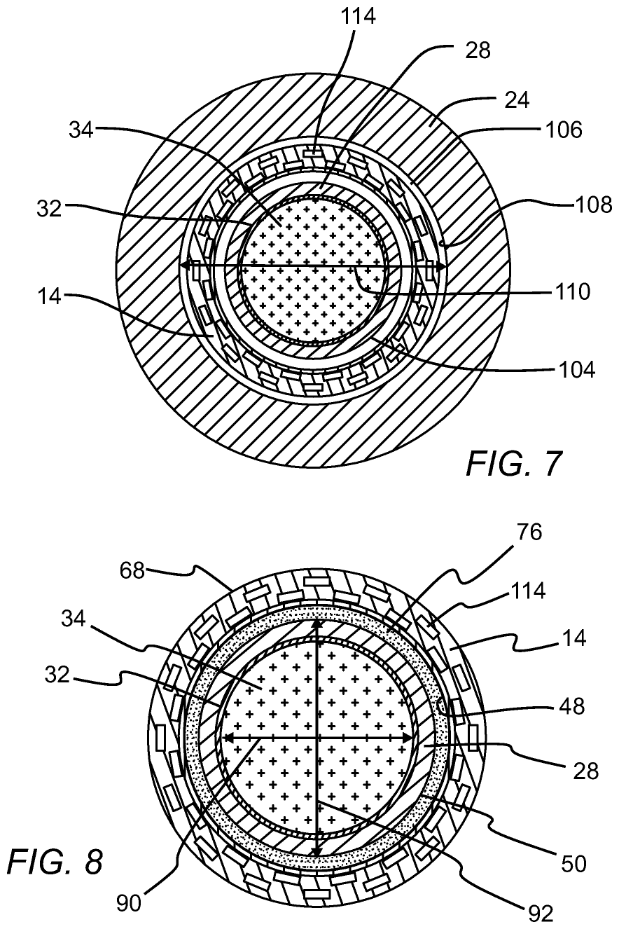

Referring to FIGS. 6-8, some embodiments of the catheter tube 28 may have an inner diameter 90 of about 0.043 inches, an outer diameter 92 of about 0.053 inches, and a corresponding wall thickness 100 of about 0.005 inches. A respective outer jacket embodiment 14 may have an inner diameter 96 of about 0.055 inches, an outer diameter 94 of about 0.063 inches, and a corresponding wall thickness 102 of about 0.004 inches. Hence a ratio of the outer jacket wall thickness 102 to the catheter tube wall thickness 100 may be about 0.8 to about 1.2. For some laser ablation catheter embodiments 12 discussed herein, the wall thickness 102 of the outer jacket 14 may be less than the wall thickness 100 of the catheter tube 28 generally such that the laser ablation catheter 12 maintains a relatively low profile along its axial length 70.

The outer jacket 14 may include the tubular jacket body 68 that may optionally have a longitudinal stiffness greater than a longitudinal stiffness of the catheter tube 28 at the proximal portion 54 (see FIG. 10) of the catheter tube 28. The longitudinal stiffness of the outer jacket 14 may be made greater than the longitudinal stiffness of the catheter tube 28 in some cases by inserting structural reinforcement into the jacket body 68, by utilizing a jacket body 68 material that has a high flexural modulus, and/or by incorporating an increased wall thickness into a proximal portion 74 of the jacket body 68. All of these features of outer jacket 14 may also act to increase the crush resistance of the outer jacket 14 if a radial load is applied to the outer jacket 14 by a user.

In some cases, the outer jacket embodiment 14 may include a reinforcement 114 that is crush resistant and that is disposed along the tubular jacket body 68 as depicted in FIGS. 11 and 12. The outer jacket 14 may have a proximal end 113 that may be disposed at or adjacent a proximal portion 54 of the catheter tube 28 of a respective laser ablation catheter embodiment and a distal end 56 that may be disposed at or adjacent the distal end 44 of the catheter tube 28.

In some cases, the reinforcement 114 may act to increase the hoop strength of the respective outer jacket 14. In general, the jacket body 68 and reinforcement 114 may also be configured in dimensions, materials, and features that increase the longitudinal stiffness and crush resistance of a respective laser ablation catheter embodiment 12. Some reinforcement embodiments 114 may include coils or braids which provide axial stiffness and crush resistance to the outer jacket 14, while still allowing for flexibility of the outer jacket 14 and respective laser ablation catheter 12 during an ablation procedure.

Referring again to FIGS. 11 and 12, in some cases, the reinforcement 114 may be disposed between layers of materials of the jacket body 68 of outer jacket embodiments 14. The jacket body 68 may include an inner jacket layer 138 with the reinforcement 114 being disposed on an outer surface 142 of the inner jacket layer 138. A tubular outer jacket layer 144 may in turn be disposed over and cover the reinforcement 114. In some cases, the inner jacket layer 138 and the outer jacket layer 144 may be formed from the same material. In some cases, the inner jacket layer 138 may be made from a first material and the outer jacket layer 144 may be made from a second material which is different from the first material. In some cases, it may be desirable for the first material of the inner jacket layer 138 to have a low coefficient of friction to facilitate assembly of the outer jacket 14 with the catheter tube 28. For some embodiments, the first material of the inner jacket layer 138 may include nylon, polyether block amides including Pebax®, polyurethanes including Pellathane®, or the like. The first material of the inner jacket layer 138 may also include fluoropolymers such as PCTFE, ETFE, EFEP, or the like that may be particularly resistant to certain sterilization methods such as radiation sterilization methods. In some instances, the first material of the inner jacket layer 138 may also include PTFE/polyimide hybrid materials. For some embodiments, a tubular structure of the inner jacket layer 138 may have a wall thickness of about 0.0005 inches or less.

For some embodiments of the outer jacket 14, the first material of the inner jacket layer 138 may be made from a lubricious polymer such as a polyimide-PTFE hybrid material or may be made from polymers that include a lubricious low friction additive such as PTFE powders, FEP powders, HDPE powders and the like. In some cases, the lubricious low friction additive known by the trade name Mobilize manufactured by Compounding Solutions, 258 Goddard Road, Lewiston, Me. may be included with the first material of the inner jacket layer 138. For some embodiments, the first material of the inner jacket layer 138 and second material of the outer jacket layer 144 may include nylons, polyether block amides including Pebax®, polyurethanes including Pellethane® or the like. The first material of the inner jacket layer 138 and second material of the outer jacket layer 144 may also include fluoropolymers such as ETFE, PCTFE and the like. The Shore hardness of the first material and the second material, particularly those that include Pebax®, may be about 70D Shore hardness to about 74D Shore hardness in some cases. For some embodiments, portions of the inner jacket layer 138 may be thermally fused or adhesively bonded to respective portions of the outer jacket layer 144. Thus, the filament structure of the reinforcement 114 may be enclosed or otherwise encapsulated by material of the jacket body 116.

FIGS. 13-14A depict an embodiment of the reinforcement 114 which is configured as a plurality of reinforcement ribbons 174 which are woven together into a tubular braid 176. In some instances, each reinforcement ribbon 174 may have a substantially rectangular transverse cross section. For some embodiments a thickness 178 of each reinforcement ribbon 174 may be from about 0.0002 inches to about 0.010 inches, and a width 180 of each reinforcement ribbon 174 may be from about 0.002 inches to about 0.005 inches (see FIG. 14A). For some embodiments, the tubular braid 176 may have a braid inner diameter 182 of about 0.001 inches larger to about 0.004 inches larger than the outer diameter 92 of the catheter tube 28.

The reinforcement ribbon 174 may be formed from any suitable flexible resilient material, for example the reinforcement ribbon 174 could be formed from a metal such as stainless steel, superelastic alloys such as nickel titanium alloy or the like. For some embodiments the reinforcement ribbon 174 may be formed from a polymer such as polycarbonate, Kevlar® para-aramid synthetic fiber material or from any suitable natural protein fiber such as silk or the like.

FIGS. 15-15A depict an embodiment of a reinforcement 114′ which is configured as a reinforcement wire 152 which is formed into a tubular coil 154, with the reinforcement wire 152 having a substantially round transverse cross section for the embodiment shown. For some embodiments, a diameter 156 of the reinforcement wire 152 may be about 0.0002 inches to about 0.015 inches (see FIG. 15A). The tubular coil 154 may have a coil inner diameter 158 of about 0.001 inches to about 0.004 inches larger than the outer diameter 92 of the catheter tube 28. The reinforcement wire 152 may be formed from any suitable high strength, flexible and/or resilient material. For example, the reinforcement wire 152 may be formed from a metal such as stainless steel, superelastic alloys such as nickel titanium alloy or the like. For some embodiments the reinforcement wire 152 may be formed from a polymer such as polycarbonate, Kevlar® para-aramid synthetic fiber material or from any suitable natural protein fiber such as silk or the like.

FIGS. 16-16A depict an embodiment of a reinforcement 114″ which is configured as a reinforcement ribbon 162 which is formed into a tubular coil 164, with the reinforcement ribbon 162 having a substantially rectangular transverse cross section. For some embodiments a thickness 166 of the reinforcement ribbon 162 may be from about 0.0002 inches to about 0.01 inches and a width 168 of the reinforcement ribbon 162 may be from about 0.002 inches to about 0.005 inches (see FIG. 16A). The tubular coil 164 may have a coil inner diameter 170 of about 0.001 inches to about 0.004 inches larger than the outer diameter 92 of the catheter tube 28. The reinforcement ribbon 162 may be formed from any suitable flexible resilient material. For example, the reinforcement ribbon 162 may be formed from a metal such as stainless steel, superelastic alloys such as nickel titanium alloy, or the like. For some embodiments the reinforcement ribbon 162 may be formed from a polymer such as polycarbonate, Kevlar® para-aramid synthetic fiber material or from any suitable natural protein fiber such as silk.

In use, embodiments of the laser ablation catheter 12 may be guided to a suitable treatment site within a patient's body by a variety of techniques. FIG. 20 illustrates the laser ablation catheter 12 emitting high energy UV laser energy 21 into a blockage 25 disposed within a body lumen 27 of a vessel 31 of a patient. For the process shown in FIG. 20, the vessel 31 may be an artery of the patient that may include a coronary artery of the patient. The laser energy 21 being emitted from the distal surface 45 of the distal optical window 40 of the laser ablation catheter 12 is high energy pulsed UV laser energy 21 which may optionally have sufficient energy fluence for photo ablation of the material of the blockage 25. However, any suitable fluence of laser energy 21 may be used depending on the indication. The distal end of the laser ablation catheter 12 is also shown being guided by an optional support catheter 24 that may be used to direct the laser ablation catheter 12 generally to the treatment site as well as to direct the distal end of the laser ablation catheter 12 to target tissue, specifically, the blockage 25.

Such a procedure may be initiated by introducing a guide sheath into the patient's femoral artery, or any other suitable vessel access site, by methods such as the Seldinger technique or any other suitable method. A guidewire 35 (see FIG. 21) which may be configured with a length, flexibility and outer diameter suitable for the intended indication, such as a coronary guidewire 35 may then be introduced into the guide sheath. Guidewire 35 may then be distally advanced through the guide sheath to the intended treatment site. Once so positioned, the support catheter 24 may then be distally advanced through the guide sheath and over the guidewire 35 to an axial position wherein a distal end of the support catheter 24 is disposed adjacent the target tissue as shown in FIG. 20. The guidewire 35 may then be proximally withdrawn from the support catheter 24 leaving an access conduit within the support catheter 24 from a position outside the patient's body to a position adjacent the blockage 25 at the treatment site. The laser ablation catheter 12 may then be distally advanced through the inner lumen of the support catheter 24 until the distal end of the laser ablation catheter 12 is disposed adjacent the blockage 25 as shown in FIG. 20. Laser energy 21 sufficient for ablation of the blockage 25 may then be transmitted into the input surface of the proximal optical window 46 as shown in FIG. 10C, and the laser ablation catheter 12 advanced into the blockage 25 while ablating material of the blockage 25 with the laser energy 21 sufficient for blockage ablation being emitted from the distal surface 45 of the distal optical window 40.

Any of the suitable laser ablation catheter embodiments discussed herein may include an eccentric guidewire lumen that may be used to slidingly accommodate a guidewire 35 that may be useful in some situations for guidance of suitable laser ablation catheter embodiments. FIGS. 17-19 illustrate an embodiment of laser ablation catheter 12′ that includes an eccentric guidewire lumen 130. In some cases, such an eccentric guidewire lumen 130 may be disposed adjacent an outer surface of the outer jacket 14′ and may be configured to slidingly accommodate a guidewire 35 as shown in FIG. 21. The laser ablation catheter embodiment 12′ may generally have any features, dimensions or materials which are the same as or similar to those of the laser ablation catheter embodiment 12 discussed herein. In some instances, the eccentric guidewire lumen 130 may include a proximal port 132 and a distal port 134 with the distal port 134 being disposed adjacent the distal end 136 of the laser ablation catheter 12′.

For the embodiment shown, the eccentric guidewire lumen 130 may include a nominal longitudinal axis 139 extending along a distal section of the laser ablation catheter 12′. In some cases, this nominal longitudinal axis 139 may be parallel to or substantially parallel to a longitudinal axis 140 of the catheter tube 28 (shown in FIG. 19) of the laser ablation catheter embodiment 12′. The laser ablation catheter embodiment 12′ may also include a distal housing 37″ with a primary lumen 148 which has a longitudinal axis 150 that is disposed concentrically with the longitudinal axis 140 of the catheter tube 28 and which is also configured to accommodate the distal optical window 40. The primary lumen 148 of the distal housing embodiment 37″ may optionally be configured to provide a crimp joint between the distal housing 37″, the catheter tube 28, the distal optical window 40 and, in some cases, the distal end 156 of the outer jacket 14′ as shown in FIG. 19. Such crimp joint embodiments may be formed as described above with regard to distal housing embodiment 37′ or the like.

The distal housing 37″ also includes an eccentric passage 149 which is disposed adjacent to the primary lumen 148 and which is configured to accommodate the eccentric guidewire lumen 130 and components thereof such as the guidewire lumen sleeve 145 which may be disposed within eccentric passage 149. The guidewire lumen sleeve 145 may also terminate distally at a distal end of the eccentric passage 149 such that the respective distal ends of the guidewire lumen sleeve 145 and eccentric passage 149 are axially coextensive.

For some embodiments, the eccentric passage 149 may be disposed within an eccentric abutment structure 146 of the distal housing 37″. The eccentric abutment structure 146 extends radially outward from a nominal tubular surface surrounding the primary lumen 148 of the distal housing 37″. The distal housing 37″ may also optionally include a proximal cylindrical section 147 that is disposed proximal to the eccentric abutment structure 146 of the eccentric passage 149. The proximal cylindrical section 147 may have a generally circular outer cross section profile free of any eccentric abutment structure and may provide, in some circumstances, a symmetric cylindrical cross section that is amenable to inwardly crimping an outer surface thereof. The distal housing embodiment 37″ may be made from a crimpable material such as stainless steel, nickel titanium alloy, titanium, or the like.

In some cases, the eccentric passage 149 of the distal housing 37″ may have a discharge axis 139′ that may be angled towards the longitudinal axis 140 of the catheter tube and/or longitudinal axis 150 of the primary lumen 148. The discharge axis 139′ may include the longitudinal axis of the eccentric passage 149 of the distal housing 37″ irrespective of the orientation of the nominal longitudinal axis 139 of the eccentric guidewire lumen 130 disposed proximally of the distal housing 37″. That is, the angular orientation of the eccentric passage 149 may differ from the angular orientation of the nominal eccentric guidewire lumen 130. In some cases, wherein the nominal longitudinal axis 139 of the eccentric guidewire lumen 130 is parallel to the longitudinal axis 140 of the catheter tube 28, the discharge axis 139′ may form an angle 151 with the nominal longitudinal axis 139 of the eccentric guidewire lumen 130 of up to about 5 degrees. In some cases, the discharge axis 139′ may form an angle 151 with the nominal longitudinal axis 139 of the eccentric guidewire lumen 130 of up to about 2.5 degrees. In some cases, the discharge axis 139′ may form an angle 151 with the nominal longitudinal axis 139 of the eccentric guidewire lumen 130 of about 2 degrees to about 5 degrees.

In some instances, the distal port 134 of the eccentric guidewire lumen 130 may be disposed about 0 mm to about 2 mm proximally from the distal end 45 of the distal optical window 40 or about 0 mm to about 2 mm proximally from the distal end 136 of the laser ablation catheter 12′ generally. For some embodiments, the proximal port 132 may be disposed about 150 mm to about 400 mm proximally from the distal end 45 of the distal optical window 40 or from the distal end 136 of the laser ablation catheter 12′ generally. In some cases, the proximal port may be disposed about 150 mm to about 250 mm from the distal end 45 of the laser ablation catheter 12′, more specifically, about 150 mm to about 200 mm from the distal end 45 of the laser ablation catheter 12′.

For some embodiments, the eccentric guidewire lumen 130 may be surrounded by a guidewire lumen sleeve 145 that extends axially from the proximal port 132 to the distal port 134. The guidewire lumen sleeve 145 may be made from a low friction material which is secured to the outer jacket 14′ with a guidewire base 143 that may include a polymer or other material similar to that of the outer jacket layer 144 of the outer jacket 14′. The guidewire base 143 may serve to bond the guidewire lumen sleeve 145 to the outer jacket 14′ and provide a smooth outer contour at the junction between the guidewire lumen sleeve 145 and the outer jacket layer 144 of the outer jacket 14′.

As discussed above, laser ablation catheters embodiments which are configured with an outer jacket can be manufactured such that the waveguide and associated materials (catheter tube and the inner layer) may be thermally cycled separately from the other components of the laser ablation catheter embodiments. Thus, the laser ablation catheter 12 (or laser ablation catheter 12′) which is configured with an outer jacket 14 (or outer jacket 14′) may be made as follows. The inside surface 33 of the catheter tube 28 may be coated with an optical coating having a first index of refraction. An appropriate thermal cycle may then be applied to the catheter tube 28 and the optical coating to adhere the optical coating to the inner surface 33 of the catheter tube 28 thereby forming the inner layer 32.

An elongated distal optical window 40 may then be attached to a distal portion 61 of the catheter tube 28. The catheter tube 28 may then be filled with a biocompatible ultraviolet transparent optical fluid 34 having a second index of refraction. A high energy laser coupler 26 may then be attached to a proximal portion 54 of the catheter tube 28 with the laser coupler 26 having a laser coupler body, a window connector body being disposed within the optical coupler body, and the proximal optical window 46 disposed within and secured to the window connector body with the proximal optical window 46 being in optical communication with the optical fluid 34 as shown in FIG. 10C. Laser energy 21 may then be transmitted through the optical fluid 34 in order to determine the optical performance of the optical coating/inner layer 32. The outer jacket 14 may then be slid over the catheter tube 28.

The outer jacket 14 may include the inner lumen 88 having the inner surface 48 which is configured to slide over an outer surface 50 of the catheter tube 28 with a close fit therebetween. In some cases, the close fit between the outer surface 50 of the catheter tube 28 and the inner surface 48 of the inner lumen 88 of the outer jacket 14 includes a dimensional clearance 98 (see FIG. 6) of about 0.0005 inches to about 0.004 inches.

The outer jacket 14 or any other suitable outer jacket embodiment, such as outer jacket 14′, discussed herein may then be secured to the catheter tube 28 such that the outer jacket 14 remains substantially fixed in relation to the longitudinal axis 60 the catheter tube 28, with the outer jacket 14 being configured to increase the longitudinal stiffness and crush resistance of the laser ablation catheter 12. The proximal portion 74 of the outer jacket 14 may be adhesively bonded to the catheter tube 28 thereby forming the proximal bond section 78 utilizing any suitable adhesive 76 such as cyanoacrylate, epoxy, U.V. cured adhesives, or the like. The distal portion 64 of the outer jacket 14 may be adhesively bonded to the catheter tube 28 thereby forming a distal bond section 79 utilizing any suitable adhesive 76 such as cyanoacrylate, epoxy, U.V. cured adhesives or the like. For some embodiments the axial length 81 of the proximal bond section 78 may be about 0.039 inches to about 3 inches (see FIG. 10 for reference), and the axial length of the distal bond section 79 measured proximally from the distal end 56 of the outer jacket 14 may be about 0.039 inches to about 0.16 inches.

Similar to the method description above with regard to use of laser catheter embodiment 12, in use, embodiments of the laser ablation catheter 12′ may be guided to a suitable treatment site within a patient's body by a variety of techniques. FIG. 21 illustrates the laser ablation catheter 12′ emitting high energy UV laser energy 21 into the blockage 25 disposed within the body lumen 27 of the vessel 31 of a patient. For the process shown in FIG. 21, the vessel 31 may be an artery of the patient that may include a coronary artery of the patient. The laser energy 21 being emitted from the distal surface 45 of the distal optical window 40 of the laser ablation catheter 12′ may optionally include high energy pulsed UV laser energy 21 with sufficient energy fluence for photo ablation of the material of the blockage 25, however, any suitable fluence of laser energy 21 may be used depending on the indication. The distal end of the laser ablation catheter 12′ may also be guided to a certain degree by the guidewire 35 disposed within the eccentric guidewire lumen 130. The guidewire 35 may be used to direct the laser ablation catheter 12′ generally to the treatment site as well as directing the distal end of the laser ablation catheter 12′ to target tissue, specifically, the blockage 25.

As discussed above, such a procedure may be initiated by introducing a guide sheath into the patient's femoral artery, or any other suitable vessel access site, by methods such as the Seldinger technique or any other suitable method. The guidewire 35 which may be configured with a length, flexibility and outer diameter suitable for the intended indication, such as a coronary guidewire 35 may then be introduced into the guide sheath. Guidewire 35 may then be distally advanced through the guide sheath to the intended treatment site. The laser ablation catheter 12′ may then be distally advanced through the guide sheath and over the guidewire 35 with the guidewire 35 disposed within eccentric guidewire lumen 130 until the distal end of the laser ablation catheter 12′ is disposed adjacent the blockage 25 as shown in FIG. 21. Laser energy 21 sufficient for ablation or other treatment of the blockage 25 may then be transmitted into the input surface of the proximal optical window 46 of laser ablation catheter 12′ as shown in FIG. 10C. The laser ablation catheter 12′ may then be advanced into the blockage 25 while ablating material of the blockage 25 with the laser energy 21 sufficient for blockage ablation being emitted from the distal surface 45 of the distal optical window 40.

Embodiments illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation and use of such terms and expressions do not exclude any equivalents of the features shown and described or portions thereof, and various modifications are possible. The term “a” or “an” can refer to one of or a plurality of the elements it modifies (e.g., “a reagent” can mean one or more reagents) unless it is contextually clear either one of the elements or more than one of the elements is described. Thus, it should be understood that although embodiments have been specifically disclosed by representative embodiments and optional features, modification and variation of the concepts herein disclosed may be resorted to by those skilled in the art, and such modifications and variations are considered within the scope of this disclosure.

With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments of the invention. Accordingly, it is not intended that the invention be limited by the forgoing detailed description.

What is claimed is:

1. A laser ablation catheter to ablate blockages in body lumens using high energy and high-power (UV) laser pulses, comprising:
a liquid filled waveguide including a catheter tube having an inner layer with a first index of refraction and a biocompatible ultraviolet transparent optical fluid disposed within and completely filling an inner lumen of the catheter tube, with the optical fluid having a second index of refraction which is greater than the first index of refraction; and
an ultraviolet grade elongated distal optical window disposed in liquid sealed relation to an inner surface of the catheter tube at a distal end of the catheter tube and in optical communication with the optical fluid;
a proximal optical window disposed in a liquid sealed relation to the inner surface of the catheter tube at a proximal end of the catheter tube and in optical communication with the optical fluid; and
an outer jacket that is disposed over the surface of the catheter tube and that has a proximal end that is disposed at a proximal portion of the catheter tube and a distal end that is disposed at a distal end of the catheter tube with the outer jacket being secured relative to the catheter tube such that the outer jacket remains substantially fixed along a longitudinal axis thereof relative to the catheter tube, the outer jacket comprising:
a tubular configuration having an inner lumen with an inner surface, with the inner surface of the inner lumen being configured to slide over an exterior surface of the catheter tube with a close fit therebetween;
a tubular jacket body that includes an inner jacket layer comprised of a first material, an outer jacket layer comprised of a second material, and a longitudinal stiffness greater than a longitudinal stiffness of the catheter tube at the proximal portion of the catheter tube, and
a reinforcement that is crush resistant and disposed between the first layer and second layer of the tubular jacket body with the tubular jacket body and reinforcement being configured to increase the stiffness and crush resistance of the laser ablation catheter.

2-5. (canceled)

6. The laser ablation catheter of claim 1 wherein the first material of the inner jacket layer comprises polyether block amide.

7. The laser ablation catheter of claim 6 wherein the first material of the inner jacket layer comprises a lubricious additive.

8. (canceled)

9. The laser ablation catheter of claim 1 wherein the first material of the inner jacket layer comprises a PTFE/polyimide hybrid material.

10-27. (canceled)

28. The laser ablation catheter of claim 1 wherein the catheter tube is secured to the distal optical window with an adhesive bond and with a distal end of the catheter tube disposed proximally of the distal end of the distal optical window. 29-30. (canceled)

31. The laser ablation catheter of claim 28 wherein a distal end of the outer jacket is secured to an outside surface of a distal portion of the catheter tube with the distal end of the outer jacket disposed proximally of the distal end of the catheter tube.

32. The laser ablation catheter of claim 31 wherein the distal end of the outer jacket is disposed about 1 mm to about 2 mm proximally of the distal end of the catheter tube.

33-43. (canceled)

44. The laser ablation catheter of claim 1 further comprising a distal housing which is secured to an outer surface of the distal end of the catheter tube with a crimped joint, the distal housing comprising a crimpable metal, a rounded bullet shaped outer surface and a stepped inner lumen with an inner surface configured to have a close fit with an outside surface of the distal end of the distal optical window and an outer surface of the distal end of the catheter tube prior to crimping.

45-57. (canceled)

58. The laser ablation catheter of claim 1 further comprising an eccentric guidewire lumen disposed adjacent an outer surface of the outer jacket, the eccentric guidewire lumen including a proximal port and a distal port which is disposed adjacent the distal end of the laser ablation catheter.

59. The laser ablation catheter of claim 58 wherein the eccentric guidewire lumen further includes a longitudinal axis that is substantially parallel to a longitudinal axis of the catheter tube.

60. The laser ablation catheter of claim 58 wherein the distal port is disposed about 0 mm to about 2 mm from the distal end of the distal optical window.

61-69. (canceled)

70. A laser ablation catheter, comprising:
a liquid filled waveguide including:
a catheter tube having an inner layer with a first index of refraction,
an optical fluid disposed within and completely filling an inner lumen of the catheter tube, with the optical fluid having a second index of refraction which is greater than the first index of refraction,
a distal optical window in optical communication with the optical fluid and disposed in sealed relation to an inner surface of the catheter tube at a distal end of the catheter tube, and
a proximal optical window in optical communication with the optical fluid and disposed in a liquid sealed relation to the inner surface of the catheter tube at a proximal end of the catheter tube;
an outer jacket that is disposed over an outer surface of the catheter tube with a proximal end that is disposed at a proximal portion of the catheter tube and a distal end that is disposed at a distal end of the catheter tube, the outer jacket comprising:
a tubular configuration having an inner lumen with an inner surface, the inner surface of the inner lumen being configured to slide over an outer surface of the catheter tube with a close fit therebetween;
a tubular jacket body that has a longitudinal stiffness greater than a proximal portion of the catheter tube and that includes an inner jacket layer comprised of a first material and an outer jacket layer comprised of a second material, and
a reinforcement that is crush resistant and disposed between the first layer and second layer of the tubular jacket body;
an eccentric guidewire lumen disposed adjacent an outer surface of the outer jacket, the eccentric guidewire lumen including a proximal port and a distal port which is disposed adjacent the distal end of the laser ablation catheter; and
a distal housing having a primary lumen which has a longitudinal axis that is concentric with a longitudinal axis of the catheter tube and which is configured to accommodate the distal optical window and an eccentric passage which is disposed adjacent the primary lumen and which is configured to accommodate the eccentric guidewire lumen.

71. The laser ablation catheter of claim 70 wherein the eccentric passage of the distal housing has a discharge axis that is angled towards the longitudinal axis of the catheter tube.

72. The laser ablation catheter of claim 71 wherein a nominal longitudinal axis of the eccentric guidewire lumen is parallel to the longitudinal axis of the catheter tube and the discharge axis forms an angle with the nominal longitudinal axis of up to about 5 degrees.

73-79. (canceled)

80. The laser ablation catheter of claim 70 wherein the distal housing is secured to an outer surface of the distal end of the catheter tube and an outer surface of a distal end of the outer jacket with a crimped joint, the distal housing comprising a crimpable metal, a rounded bullet shaped outer surface and a stepped inner lumen with an inner surface configured to have a close fit with an outside surface of the distal end of the distal optical window, an outer surface of the distal end of the catheter tube and an outer surface of the distal end of the outer jacket prior to crimping.

81. (canceled)

82. The laser ablation catheter of claim 70 wherein the proximal port is disposed about 150 mm to about 250 mm from the distal end of the distal optical window.

83-84. (canceled)

85. A laser ablation catheter, comprising:
a liquid filled waveguide including:
a catheter tube having an inner layer with a first index of refraction,
an optical fluid disposed within and completely filling an inner lumen of the catheter tube, with the optical fluid having a second index of refraction which is greater than the first index of refraction,
a distal optical window in optical communication with the optical fluid and disposed in sealed relation to an inner surface of the catheter tube at a distal end of the catheter tube, and
a proximal optical window in optical communication with the optical fluid and disposed in a liquid sealed relation to the inner surface of the catheter tube at a proximal end of the catheter tube; and
an outer jacket that is disposed over an outer surface of the catheter tube with a proximal end that is disposed at a proximal portion of the catheter tube and a distal end that is disposed at a distal end of the catheter tube, the outer jacket comprising:
a tubular configuration having an inner lumen with an inner surface, the inner surface of the inner lumen being configured to slide over an outer surface of the catheter tube with a close fit therebetween;
a tubular jacket body that has a longitudinal stiffness greater than a proximal portion of the catheter tube and that includes an inner jacket layer comprised of a first material and an outer jacket layer comprised of a second material, and
a reinforcement that is crush resistant and disposed between the first layer and second layer of the tubular jacket body.

86. The laser ablation catheter of claim 85 further comprising a distal housing crimped onto a distal end of the catheter tube and the distal optical window.

87. The laser ablation catheter of claim 86 wherein the distal housing is secured to an outer surface of the distal end of the catheter tube and an outer surface of a distal end of the outer jacket with a crimped joint, the distal housing comprising a crimpable metal, a rounded bullet shaped outer surface and a stepped inner lumen with an inner surface configured to have a close fit with an outside surface of the distal end of the distal optical window, an outer surface of the distal end of the catheter tube and an outer surface of the distal end of the outer jacket prior to crimping.

88-90. (canceled)

91. The laser ablation catheter of claim 85 further comprising a proximal inner sleeve that has a tubular configuration, that is disposed over the catheter tube, that axially overlaps a proximal end of the outer jacket and that extends proximally to the proximal optical window.

92-95. (canceled)
